(19) United States
(12) Patent Application Publication
(10) Pub. No.: US 2022/0110791 A1
(43) Pub. Date: Apr. 14, 2022

(54) DEVICES AND METHODS FOR CAPSULE SHIELDING IN CATARACT SURGERY

(71) Applicant: MOR RESEARCH APPLICATIONS LTD., Tel Aviv (IL)

(72) Inventors: Tova LIFSHITZ, Tel Aviv (IL); Nadav Yaakov BELFAIR, Tel Aviv (IL); Eliyahu BSHARIM, Tel Aviv (IL)

(21) Appl. No.: 17/430,449

(22) PCT Filed: Mar. 4, 2020
(86) PCT No.: PCT/IB2020/051859

§ 371 Date: Aug. 12, 2021

(30) Foreign Application Priority Data
Mar. 4, 2019 (IB) PCT/IB2019/051715

(51) Int. Cl. A61F 9/007

(52) U.S. Cl. CPC A61F 9/00745

(57) ABSTRACT

Aspects of embodiments pertain to a lens capsule shielding device for cataract surgery, the shielding device comprising a handle and at least one shielding element slidably mounted inside the handle. The shielding element is selectively configurable in an deployed configuration and a retracted configuration, wherein the at least one shielding element, in the deployed configuration, is operable to be positionable between lens portions of the patient's eye and posterior lens capsule portion of the eye and configured to function as a shield for preventing damage of the posterior lens capsule portion during the application of energy for fragmenting lens portions and/or for preventing damage from suction forces applied for the removal of lens fragments.

### BACKGROUND

During phacoemulsification cataract surgery, the surgeon employs a phacoemulsification (Phaco) device to disintegrate the lens and apply suction to extract the lens fragments through the Phaco device.

It is important to preserve enough distance between the Phaco tip and the posterior capsule of lens to prevent inadvertent suction to the capsule thereby rupturing it causing serious complications to the entire procedure. This complication can occur at any stage of the phacoemulsification; however, the final stages are particularly prone because of the need to further fragmentize remainder lens nucleus pieces through the application of ultrasound (US) energy. During the fragmentation of remainder lens nucleus pieces there is no lens material present that could provide a natural shielding between the phaco tip and the posterior lens capsule while application of US energy to further fragmentize the nucleus pieces.

### BRIEF DESCRIPTION OF THE DRAWINGS

The subject matter regarded as the invention is particularly pointed out and distinctly claimed in the concluding portion of the specification. Invention components, features, their interaction, operation, and advantages are best understood with reference to the following detailed description and accompanying drawings in which:

FIGS. 1A-1C are schematic diagrams depicting lens phacoemulsification and capsule shielding, respectively, according to an embodiment.

FIGS. 2A-2C are different illustrative views of a shielding device, according to some embodiments.

FIGS. 3A-3B are different illustrative views of a shielding device, according to some other embodiments.

FIG. 4 is an illustrative view of a shielding device, according to an alternative embodiment.

It will be appreciated that for simplicity and clarity of illustration, elements shown in the figures are not necessarily drawn to scale and reference numerals may be repeated among the figures indicating corresponding or analogous elements.

### DETAILED DESCRIPTION OF EMBODIMENTS

The following description sets forth various details to provide an understanding of the invention and it should be appreciated, by those skilled in the art, that the present invention may be practiced without these specific details. Furthermore, well-known methods, procedures, and components have been omitted to highlight the present invention.

In one example, it is an object of the invention to provide a lens-capsule shielding device configured to provide shielding of the lens capsule during phacoemulsification of the lens in cataract surgery and easy retrieval following the procedure.

In some embodiments, the shielding device comprises a shielding element that is selectively interoperably expandable and retractable into a deployed and retracted configuration for facilitating lens fragmentation and suction. In some embodiments, the shielding element may be operably coupled with an actuator that is actuatable by a user for selectively deploying and retracting the shielding element. The actuator may for example comprise a mechanical, pneumatic, hydraulic and/or electromagnetic actuation arrangement. In some embodiments, the actuator may comprise a slidable drive element that is operably engagable by the user while holding the shielding device.

In the deployed configuration, the shielding element assumes an expanded shape to provide shielding functionalities. In the retracted configuration, the shielding element may assume a collapsed or reduced configuration such that the shielding element does not obstruct the view towards the posterior lens capsule. For example, prior to employing ultrasound-based lens fragmentation, a lens portion may be placed onto a shielding element of the device so that the shielding element is placed between a posterior capsule portion and one or more lens fragments. The US-energy emitting phacoemulsification tip (also: phaco tip) can access and operably engage the one or more lens fragments through the anterior chamber while the lens fragments are supported by the shielding element positioned between the phaco tip and the posterior capsule portion.

In some embodiments, shielding element may be configured to absorb energy emitted by the phacoemulsification tip without being torn and/or otherwise damaged by the emitted energy, yet the shielding element may optionally also be configured to be flexible enough to be selectively deployable into an expanded configuration and retractable into a channel to assume a collapsed configuration. In some examples, the shielding element may comprise a membrane having, e.g., lugs and cleats. The membrane may for example be made of Polymer material including, for instance, silicone.

Shielding device may include a handle for allowing gripping of the shielding device by a medical professional, and an applicator member having a proximal applicator opening. At least a portion of applicator member is safely insertable into and removable from a human eye for deploying and retracting the shielding device as disclosed herein. The term “proximal” refers to a location of the device which is closer to a patient compared to a “distal” location, during use of the device by a medical professional.

In some embodiments, the shielding element can be adjusted by increasing or decreasing shield area until the Phaco is removed. When a deployment area is selected, a locking mechanism can be engaged to secure the shielding element(s) in the desired degree of shield expansion.

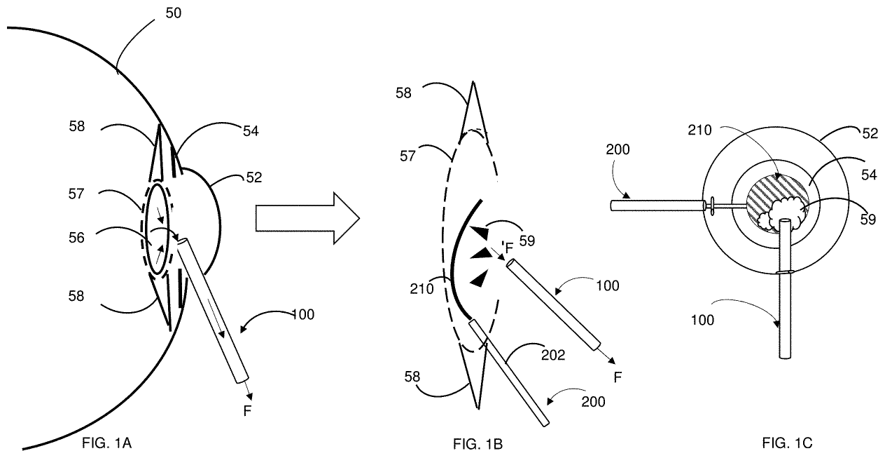

FIG. 1A is a schematic view of eye globe 50 depicting cornea 52, expanded iris 54, cloudy lens 56, lens capsule 57, and zonules 58. As shown, a Phaco device 100 traverses cornea 52 and disintegrates lens 56 through the application of ultrasound and removes lens fragments 59 through suction and pulls them through the Phaco device 100, as schematically indicated by arrow F.

FIG. 1B is schematic side view of lens 56 and lens capsule 57 held by zonules 58, and FIG. 1C is a schematic top view of lens 56. As shown, shielding element 210 can be deployed between the posterior portion of lens capsule 57 and lens fragments 59 for shielding the posterior portion of lens capsule 57 from the tip from the Phaco device 100 during further lens portions fragmentation thereby and suction of additionally splits lens portions and/or fragments 59 into Phaco device 100. This way, the risk of inadvertently subjecting the posterior portion of lens capsule 57 from otherwise potentially damaging ultrasound energy and suction may be reduced or prevented.

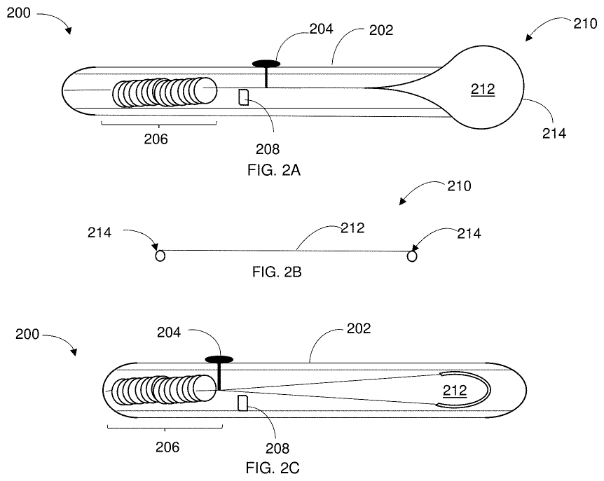

Additional reference is made to FIGS. 2A-2C. FIG. 2A is schematic, cross-sectional view of a first embodiment of lens capsule shielding device 200 in a deployment configuration. As shown, shielding device 200 may include a handle 202 and an actuator 204 that is operably coupled with shielding element 210 for selectively driving shielding element 210, e.g., out of the handle 202 or of an applicator member as described below, such that the shielding element assumes an extended or deployed position, and for retracting shielding element 210 into handle 202 to assume a retracted position in which the shielding element 210 may attain a folded or crimped configuration. Optionally, actuator 204 may comprise a drive element that is slidably mounted in handle 202 for selectively deploying from and retracting shielding element 210 back into a channel of the shielding device. Optionally, actuator 204 may comprise one or more push buttons for selectively deploying and retracting shielding element 210. Optionally, handle 202 and/or the applicator member may be channeled or otherwise configured to allow retraction and securing of shielding element 210 in the retracted configuration.

Optionally, actuator 204 may comprise a mechanical energy storage element 206 (e.g., a tension spring) operative to apply a retracting force to shielding element 210, and a locking mechanism 208 configured to secure shielding element 210 in the extended position selected by a surgeon. FIG. 2A schematically shows the shielding element 210 in the extended position and FIG. 2C schematically shows the shielding element in the retracted position. It should be appreciated that such biasing and locking schemes are also applicable to other embodiments. Various locking mechanisms frictionally and/or form-locking locking mechanism can be employed like, e.g., friction stops and/or latch mechanisms. In a certain embodiment, actuator 204 is implemented as a portion of the shielding element.

Shielding element 210 may be selectively expandable into a deployment configuration and a storage position. Shielding element 210 may include, for example, a membrane 212 (e.g., implemented by a webbing) that is controllably expandable into a deployment configuration. Optionally, shielding element 210 may comprise an (e.g., based) expandable frame 214 for spanning membrane 212 into the deployment configuration. It should be appreciated that, in certain variant embodiments, frame 214 is biased to assume a storage configuration (e.g., folded configuration) for storage inside handle 202.

FIG. 2B is a schematic cross-sectional view of shielding element 210 depicting an expanded membrane 212 and a membrane-spanning frame 214. In a certain embodiment, biased frame 214 is implemented with a metallic material like high carbon alloy steel, stainless steel, or other metallic materials having such functionality. In another embodiment, this biased frame 214 is implemented with a polymeric material. In some examples, membrane 212 may be implemented as either a silicon-based material, a hydrogel-based material, or Polymethyl methacrylate (PMMA) and/or any other suitable materials that can provide such biasing functionality.

In some embodiments, frame 214 may be implemented by smart materials such as smart memory alloys, and actuator 204 may be operable configure frame 214 such to be membrane-spanning.

In some embodiments, membrane 212 is operable to provide a shielding functionality during, e.g., phacoemulsification in cataract surgery. For example, membrane 212 has a thickness capable of reducing or prevent the danger of tearing posterior portion of lens capsule 57, e.g., due to suction, and dampen ultrasound vibration, while enabling the bias of frame 214 to drive shielding element 210 into a deployment configuration. For example, membrane 212 may be configured to allow engagement of thereof with the phaco tip during phaco emulsification without damaging membrane 212 (e.g., tearing or otherwise disintegration) due to the application of ultrasound energy and/or negative pressure/suction force. Accordingly, in some embodiments, the shielding capacity is a function of the strength and/or elasticity of the webbing material employed.

In some embodiments, shielding element 210 may assume in the expanded (also: deployed) configuration a convex shape relative to the posterior capsule portion such that the shielding element can act as a receptacle for holding lens portions during the phacoemulsification process for further fragmentation of the lens portions into smaller pieces or fragments.

As shown in FIG. 2C, in a non-deployed configuration, shielding element 210 is withdrawn into handle 202 where shielding element 210 assumes a collapsed state. It should be appreciated that in a certain embodiment, the angle between shielding element 210 and actuator 204 is predefined and is implemented through biasing frame 214 with the desired deployment angle. This configuration advantageously enables deployment of shielding element 210 while shielding device 200 is disposed in the eye. In a certain embodiment handle 202 has a diameter of 0.9 mm, however, it should be appreciated that the diameter can be implemented in accordance with various procedure requirements.

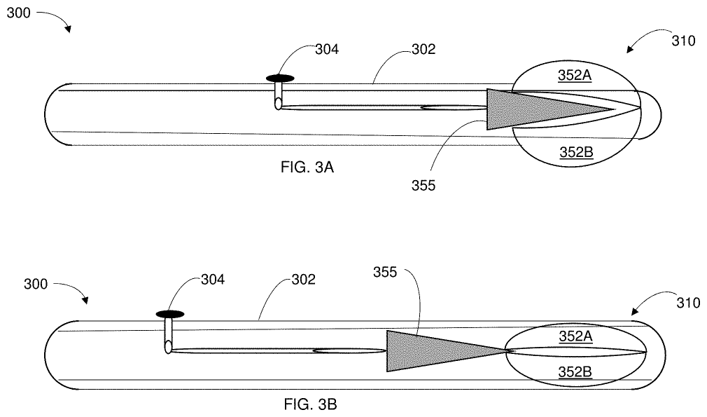

FIG. 3A is a schematic cross-sectional view of another embodiment of a lens-capsule shielding device 300 in a deployment configuration. As shown, shielding device 300 includes handle 302 with, for example, a slidably mounted, actuator 304 fitted with a deployment expander element 355 (e.g., a wedge-shaped element) operative to deploy a shielding element 310 implemented by two pivotally mounted crescent shielding components 352A and 352B through rotation around a pivotal mount. It should be appreciated that various deployment mechanisms responsive to the application of user force to a deployment mechanism are included within the scope of this application. It should be further appreciated that crescent shielding components 352A and 352B are biased to assume a closed position such that their respective concavities are facing each other. However, it should be noted that other shielding geometries set in accordance with patient requirements are also included with the scope of the present invention.

FIG. 3B depicts the embodiment of FIG. 3A in a retracted or storage configuration achieved by retracting shielding components 352A and 352B into handle 302 where shielding element 310 assumes a collapsed configuration.

It should be noted that in a certain embodiment, that webbing spans space in between crescent components 352A and 352B.

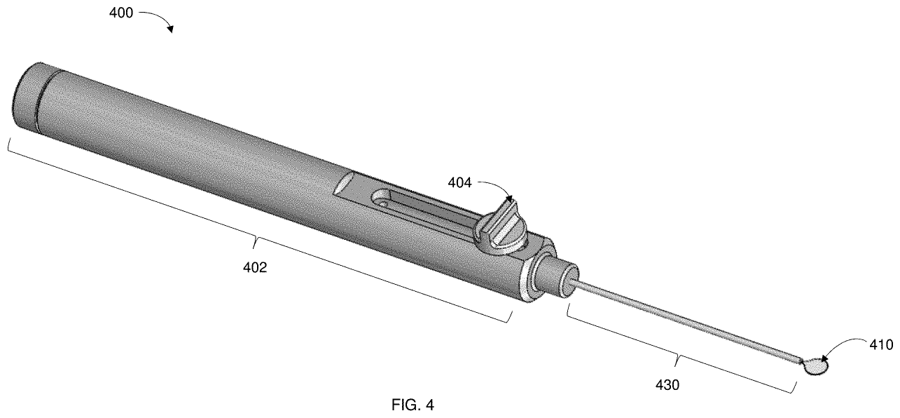

FIG. 4 depicts another embodiment of a shielding device 400 in a deployed configuration. Shielding device 400 may include a handle 402 and an actuator 404, which may comprise a slidable drive element, is operably coupled with shielding element 210 for driving shielding element 410 out of the handle 402 to assume an extended or deployed position, and for retracting shielding element 410 into handle 402 to assume a retracted position. Optionally, actuator 404 may comprise a drive element that is slidably engageable by the user and slidably coupled with handle 402. Optionally, handle 402 may be a channeled handle or otherwise configured to allow retraction and securing of shielding element 410 in the retracted configuration.

Shielding devices as disclosed herein may also include an applicator member having a diameter that is small enough such that at least a portion of the applicator member is safely insertable into a patient's eye for deployment of the shielding element. For instance, as depicted schematically in FIG. 4, an applicator member 430 may extend from the handle 402 and have a proximal opening for deployment and retraction of shielding element 410. Actuator 404 is operably coupled with shielding element 410.

Aspects of embodiments may also pertain to a method of lens capsule shielding device for use in cataract surgery. The method may comprise positioning a shielding element between lens portions of the patient's eye and posterior lens capsule portion of the eye and operable to act as a shield for preventing damage of the posterior lens capsule portion during the application of energy for fragmenting lens portions and/or for preventing damage from suction forces applied for the removal of lens fragments.

### ADDITIONAL EXAMPLES

Example 1 concerns a lens capsule shielding device for use in cataract surgery, the shielding device comprising: a handle; and at least one shielding element operably coupled with the handle such that the shielding element is selectively configurable in a deployed configuration and a retracted configuration, wherein the at least one shielding element, in the deployed configuration, is operable to be positionable between lens portions of the patient's eye and posterior lens capsule portion of the eye and operable to act as a shield for preventing damage of the posterior lens capsule portion during the application of energy for fragmenting lens portions and/or for preventing damage from suction forces applied for the removal of lens fragments.

Example 2 includes the subject matter of Example 1 and, optionally, wherein the shielding element comprises a wire frame biased to assume the deployed configuration in accordance with a degree of the shielding element being disposed outside of the handle; and a flexible shielding membrane spanning the wire frame.

Example 3 includes the subject of Example 3 and, optionally, wherein the wire frame is constructed from a metallic and/or a polymeric material.

Example 4 includes the subject matter of Examples 2 or 3 and, optionally, wherein the shielding webbing is constructed from a silicon-based material and/or hydrogel-based material and/or a Polymethyl methacrylate (PMMA)

Example 5 includes the subject matter of any one or more of the Examples 1 to 4 and, optionally, a locking mechanism configured to secure shielding element in the deployed configuration.

Example 6 includes the subject matter of Example 5 and, optionally, wherein the locking mechanism includes a latching arrangement.

Example 7 includes the subject matter of any one or more of the Examples 1 to 6 and, optionally, a mechanical energy storage element in communication with the shielding element, the mechanical energy storage element operative to apply a retraction force on the shielding element forcing the shielding element to retract into the handle.

Example 8 includes the subject matter of any one or more of the Examples 1 to 7 and, optionally, wherein the at least one shielding element includes a plurality of shielding components.

Example 9 includes the subject matter of Example 8 and, optionally, wherein the plurality of shielding components are pivotally mounted to the handle.

Example 10 includes the subject matter of Examples 8 or 9 and, optionally, wherein at least one of the plurality of shielding components is implemented with a crescent surface geometry.

Example 11 includes the subject matter of any one or more of the Examples 1 to 10 and, optionally, wherein the shielding element is sized such to be positionable between posterior portion of the lens capsule and the lens or lens fragments.

Example 12 includes the subject matter of any one or more of the Examples 1 to 11 and, optionally, wherein the shielding element is sized such to be positionable between posterior portion of the lens capsule and the lens or lens fragments during phacoemulsification

Example 13 includes the subject matter of any one or more of the Examples 1 to 12 and, optionally, further comprising an actuator, wherein the at least one shielding element operably coupled with the actuator such that the shielding element is selectively configurable in a deployed configuration and a retracted configuration responsive to operable engagement with the actuator.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A lens capsule shielding device for use in cataract surgery, the shielding device comprising:
a handle; and
at least one shielding element operably coupled with the handle such that the shielding element is selectively configurable in a deployed configuration and a retracted configuration,
wherein the at least one shielding element, in the deployed configuration, is operable to be positionable between lens portions of the patient's eye and posterior lens capsule portion of the eye and operable to act as a shield for preventing damage of the posterior lens capsule portion during the application of energy for fragmenting lens portions and/or for preventing damage from suction forces applied for the removal of lens fragments.

2. The lens capsule shielding device of claim 1, wherein the at least one shielding element comprises a wire frame biased to assume the deployed configuration in accordance with a degree of the shielding element being disposed outside of the handle; and
a shielding membrane spanning the wire frame of the at least one shielding element.

3. The shielding device of claim 2, wherein the wire frame is constructed from a metallic and/or a polymeric material.

4. The shielding device of claim 2 or claim 3, wherein the shielding membrane is constructed from a silicon-based material and/or hydrogel-based material and/or a Polymethyl methacrylate (PMMA).

5. The shielding device of any one or more of the preceding claims, further comprising a locking mechanism configured to secure shielding element in the deployed configuration.

6. The shielding device of claim 5, wherein the locking mechanism includes a latching arrangement.

7. The shielding device of any one or more of the preceding claims, further comprising a mechanical energy storage element in communication with the shielding element, the mechanical energy storage element operative to apply a retraction force on the shielding element forcing the shielding element to retract into the handle.

8. The shielding device of any one or more of the preceding claims, wherein the at least one shielding element includes a plurality of shielding components.

9. The shielding device of claim 8, wherein the plurality of shielding components is pivotally mounted to the handle.

10. The shielding device of claim 8 or claim 9, wherein at least one of the plurality of shielding components is implemented with a crescent surface geometry.

11. The shielding device of any one or more of the preceding claims, wherein the shielding element is sized such to be positionable between posterior portion of the lens capsule and the lens or lens fragments.

12. The shielding device of any one or more of the preceding claims, wherein the shielding element is sized such to be positionable between posterior portion of the lens capsule and the lens or lens fragments during phacoemulsification.

13. The shielding device of any one or more of the preceding claims, further comprising: an actuator, wherein the at least one shielding element operably coupled with the actuator such that the shielding element is selectively configurable in a deployed configuration and a retracted configuration responsive to operable engagement with the actuator.
